(19) United States
(12) Patent Application Publication
(10) Pub. No.: US 2026/0273280 A1
(43) Pub. Date: Sep. 17, 2026

(54) ENVIRONMENTAL-BASED POWER ADJUSTMENT IN AN IMPLANTABLE DEVICE

(71) Applicant: Cochlear Limited, Macquarie University, NSW (AU)

(72) Inventors: Kristian Gunnar ASNES, Molndal (SE); Johan BERGMAN, Öjersjö (SE)

(21) Appl. No.: 19/168,120

(22) PCT Filed: Apr. 1, 2024
(86) PCT No.: PCT/IB2024/053158

§ 371 Date: Sep. 23, 2025

Related U.S. Application Data
(60) Provisional application No. 63/494,935, filed on Apr. 7, 2023.

(51) Int. Cl. A61N 1/36; A61N 1/05; A61N 1/08; A61N 1/378

(52) U.S. Cl. CPC A61N 1/36038; A61N 1/0541; A61N 1/08; A61N 1/36125; A61N 1/3787

(57) ABSTRACT

Presented herein are techniques for adjusting power supplied to elements of an implantable device, such as an implantable acoustic hearing device, based on environmental conditions associated with an ambient environmental.

### BACKGROUND

### Field of the Invention

The present invention relates generally to adjusting power transmitted to an implantable device based on environmental conditions.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: determining an amplitude of an environmental signal in an environment; determining a voltage to set an amplifier of an implantable medical device based on the amplitude of the environmental signal; and adjusting a power provided to the amplifier based on the voltage.

In another aspect, one or more non-transitory computer readable storage media are provided comprising instructions. The instructions, when executed by a processor, cause the processor to: determine at least one attribute of a sound signal captured from an ambient environment; determine a voltage to set an amplifier of an implantable acoustic hearing device based on the at least one attribute of the sound signal; and adjust a power provided to the amplifier based on the voltage.

In another aspect, an implantable acoustic hearing device is provided. The implantable acoustic hearing device comprises: a battery; an amplifier; and a processor configured to set a power provided by the battery to the amplifier based on an amplitude of at least one sound signal in an ambient environment of the implantable acoustic hearing device.

In another aspect, a medical device is provided. The medical device comprises: an input configured to capture one or more spatial signals from an ambient environment; an output transducer; an amplifier configured to drive the output transducer based on the one or more spatial signals; and at least one processor configured to: determine a voltage to at which to set the amplifier based on an amplitude of the one or more spatial signals, and adjust a power provided to the amplifier based on the voltage.

### BRIEF DESCRIPTION OF THE DRAWINGS

Embodiments of the present invention are described herein in conjunction with the accompanying drawings, in which:

FIG. 1A is a schematic diagram illustrating a semi-implantable acoustic hearing device with which aspects of the techniques presented herein can be implemented;

FIG. 1B is a block diagram of an embodiment of the semi-implantable acoustic hearing device of FIG. 1A;

FIG. 1C is a block diagram of another embodiment of the semi-implantable acoustic hearing device of FIG. 1A;

FIG. 2A is a schematic diagram illustrating a totally-implantable acoustic hearing device with which aspects of the techniques presented herein can be implemented;

FIG. 2B is a block diagram of the totally-implantable acoustic hearing device of FIG. 2A;

FIG. 3 is a block diagram of a portion of an implantable acoustic hearing device with which aspects of the techniques presented herein can be implemented;

FIG. 4 shows graphs illustrating aspects of the techniques presented herein;

FIG. 5 is a flowchart illustrating a method in accordance with certain embodiments presented herein;

FIG. 6 is a schematic diagram illustrating a vestibular stimulator system with which aspects of the techniques presented herein can be implemented; and

FIG. 7 is a schematic diagram illustrating a retinal prosthesis system with which aspects of the techniques presented herein can be implemented.

### DETAILED DESCRIPTION

Presented herein are techniques for adjusting power supplied to elements of an implantable device, such as an implantable acoustic hearing device, based on environmental conditions associated with an ambient environmental. In certain arrangements, a dynamic radio frequency (RF) link power link can be used to adjust a power level transmitted from an external portion to an implantable device (e.g., an implantable acoustic hearing device). The voltage in the implantable device can be regulated by a voltage regulator to supply a voltage to an amplifier, such as a Class D amplifier. A typical Class D amplifier has an operating voltage range of close to a factor of two and since static power consumption and noise are roughly proportional to voltage squared, large reductions of the static power consumption and the noise can be achieved by using a lowest possible voltage.

In some listening environments, such as quiet environments, the target voltage to supply to the amplifier may be lower than in louder acoustic environment. When a lower voltage is supplied to the amplifier, a lower power may be transmitted to the voltage regulator to supply the lower voltage to the amplifier. By providing the acoustic implant with enough power to obtain a voltage in a lower range, significant power savings can be accomplished. In addition, lowering a drive voltage to the amplifier reduces the static noise experienced by the recipient. By lowering the drive voltage in quiet environments, the static noise is reduced in an environment where the noise is most audible since it is not masked by other sounds. Therefore, lowering the voltage supplied to the amplifier in quiet environments has the benefits of reducing both battery power consumption and static noise.

The target voltage to supply to the amplifier for an acoustic signal may be determined based on parameters of the acoustic signal. The suitable target voltage may be based on, for example, a peak value of the signal (e.g., in different frequency bands), expected power consumption, battery status, a listening program, a classifier analysis, or a combination of factors. A processor in the external portion of the implantable acoustic hearing device has access to detailed information about incoming acoustic signals and the processor is able to determine a suitable target voltage to supply to the implantable portion of the implantable acoustic hearing device based on the properties of acoustic signals. By varying the implant voltage based on parameters of received sound signals, the required power to supply to the acoustic implant to achieve the target implant voltage is also varied. Therefore, by analyzing properties of the incoming sound signals, in certain environments, the target voltage supplied to the amplifier may be lowered, and the power supplied to the voltage regulator may also be lowered.

Embodiments described herein provide for analyzing an environmental/spatial signal in the form of an acoustic sound signal, determining a target voltage needed at an amplifier to provide the acoustic signal to a recipient of an acoustic implant, and providing power based on the target voltage. In some embodiments, the target voltage may be determined based on the amplitude of the acoustic signal. In some embodiments, the target voltage may be determined based on environmental conditions associated with the recipient (e.g., when the recipient is in a quiet environment). In certain embodiments, the environmental/spatial signal could be other types of signals (e.g., light signals, etc.).

There are a number of different types of devices in/with which embodiments of the present invention may be implemented. Merely for ease of description, the techniques presented herein are primarily described with reference to a specific device in the form of a cochlear implant system. However, it is to be appreciated that the techniques presented herein may also be partially or fully implemented by any of a number of different types of devices, including consumer electronic device (e.g., mobile phones), wearable devices (e.g., smartwatches), hearing devices, implantable medical devices, wearable devices, etc. consumer electronic devices, wearable devices (e.g., smart watches, etc.), etc. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems. combinations or variations thereof, etc.) or a device for use by a person with normal hearing (e.g., consumer devices that provide audio streaming, consumer headphones, earphones and other listening devices). In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

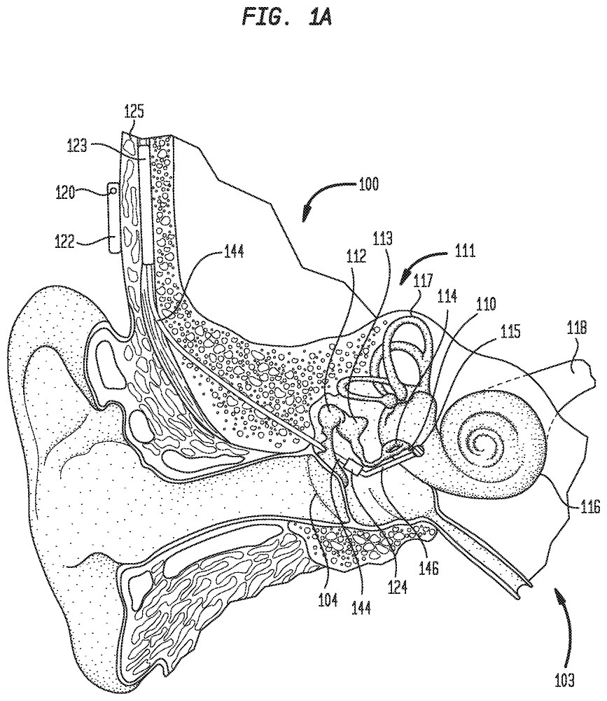

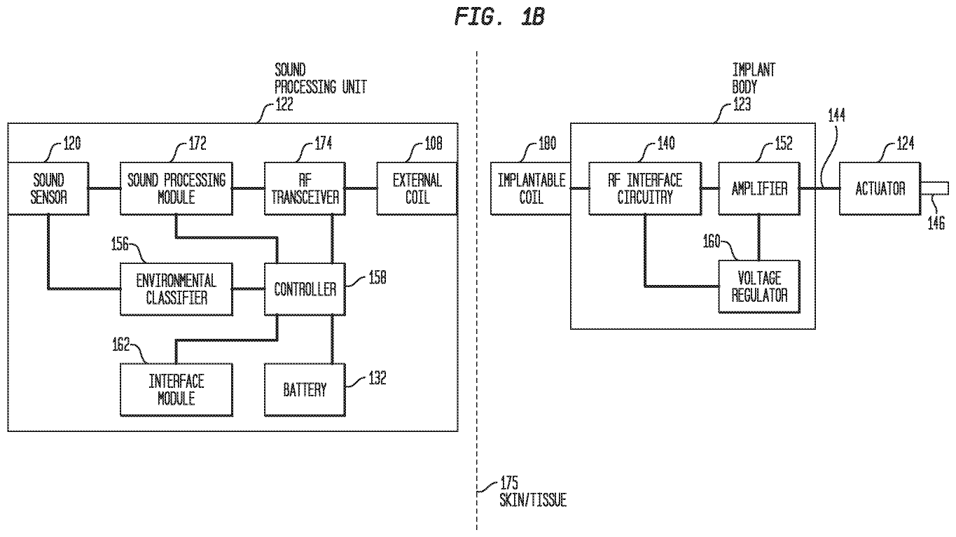

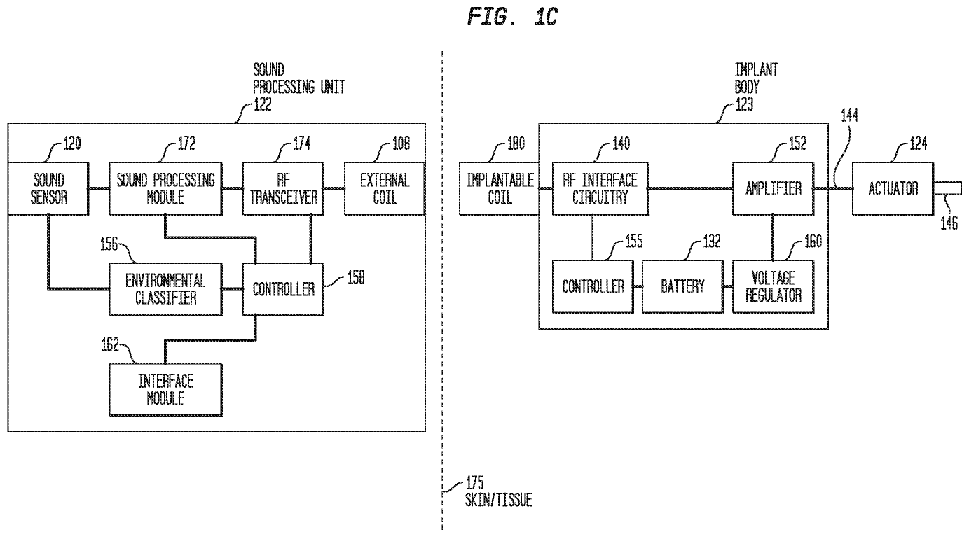

FIG. 1A is a schematic diagram illustrating an implantable acoustic hearing device 100 in accordance with certain embodiments presented herein. FIGS. 1B and 1C are block diagrams of embodiments of the implantable acoustic hearing device 100. For ease of description, FIGS. 1A-1C will be described together.

The implantable acoustic hearing device 100 is, in general, configured to convert sound signals entering the recipient's outer ear into mechanical vibrations that are directly or indirectly transferred to the cochlea 116, thereby causing generation of nerve impulses that result in the perception of the received sound.

In the embodiments of FIG. 1A, the implantable acoustic hearing device 100 includes a sound sensor 120, a sound processing unit 122, an implant body 123, and an implantable actuator/transducer 124. The sound sensor 120 and the sound processing unit 122 are external components configured to be coupled to, or worn by, the recipient, while the implant body 123 and the actuator 124 are implanted in the head 125 of the recipient. Since only some of the components of the implantable acoustic hearing device 100 are implanted within the head 125 of the recipient, the implantable acoustic hearing device 100 can be referred to as a “semi-implantable” acoustic hearing prosthesis. Although not shown in FIG. 1A, the actuator 124 can include a hermetically-sealed housing.

FIG. 1A illustrates an example in which the sound sensor 120 is an external sound sensor. In such embodiments, an external sound sensor could be worn by the recipient, located at or in the outer ear, etc. and the received sound signals could be transcutaneously transferred to the sound processing unit 122. However, it is to be appreciated that this specific configuration is illustrative and that a variety of different sound sensors and locations may be used in accordance with certain embodiments presented herein. For example, in alternative embodiments, the implantable acoustic hearing device 100 could include a sound sensor coupled to an auditory element of the recipient's ear that vibrates in response to receipt of sound signals, such as the tympanic membrane 104, the ossicles 111 (e.g., one or more of the malleus 112, the incus 113 or the stapes 114), the oval window 110, the recipient's round window 115, semicircular canals 117, etc.

The sound processing unit 122 comprises a sound processor (not shown in FIG. 1A) that converts sounds sensed by the sound sensor 120 into electrical signals that are transcutaneously transferred to the implant body 123 via a radio frequency (RF) link. The implant body 123 uses the electrical signals received from the sound processing unit 122 to drive the actuator 124. In the embodiment of FIG. 1A, the actuator 124 is mechanically coupled to the round window 115 via a coupling element 146. As such, the coupling element 146 relays the vibration generated by the actuator 124 to the round window 115, which in turn, causes round window 115 to vibrate. In an alternate embodiment, the actuator 124 could be coupled to the oval window 110, an opening in the semicircular canals 117, directly to an implant element reaching into the cochlear fluid, or another structure of the inner ear 103.

FIG. 1B illustrates an example in which the battery is located in an external portion of the implantable hearing device and FIG. 1C illustrates an example in which the battery is located in an internal portion of the implantable acoustic hearing device. As shown in FIGS. 1B and 1C, the sound processing unit 122 comprises sound sensor 120, a sound processing module 172, an environmental classifier 156, a controller (control circuit) 158, an interface module 162, an RF transceiver 174, and an external coil 108. In the embodiment illustrated in FIG. 1B, sound processing unit 122 additionally includes at least one battery 132. As shown in FIGS. 1B and 1C, implant body 123 comprises implantable coil 180, RF interface circuitry 140, amplifier 152, and voltage regulator 160 all configured to be implanted under the skin/tissue (tissue) 175 of the recipient. In the embodiment illustrated in FIG. 1C, implant body 123 additionally includes controller 155 and at least one battery 132.

In operation, the sound sensor 120 of sound processing unit 122 is configured to receive sound signals (sound) and to convert the received sound into electrical signals. If other sound input devices are present, the sound could also or alternatively may be received by as an electrical signal. As shown in FIGS. 1B and 1C, electrical signals are output by sound sensor 120 to a sound processing module 172. The sound processing module 172 is configured to convert the electrical signals into adjusted/processed electrical signals. That is, the sound processing module 172 is configured to apply one or more processing operations (e.g., filtering, noise reduction, automatic gain control/adjustment, loudness compression, etc.) to the electrical signals. In certain embodiments, the sound processing module 172 may include a digital signal processor.

Implantable acoustic hearing device 100 comprises at least one battery 132. In the embodiment illustrated in FIG. 1B, the at least one battery 132 is included in sound processing unit 122. In the embodiment illustrated in FIG. 1C, the at least one battery 132 is included in implant body 123. The at least one battery 132 provides electrical power to the various components of implantable acoustic hearing device 100. Battery 132 may be used to supply power to any electrically powered circuits/components of implantable acoustic hearing device 100, including controller 158, sound processing module 172, amplifier 152, actuator 124, etc. In one embodiment, the at least one battery provides electrical power to the voltage regulator 160 to drive amplifier 152 to a target voltage for amplifying an acoustic signal.

Sound processing unit 122 includes an environmental classification module (environmental classifier) 156. As shown, the environmental classifier 156 receives the electrical signals output by the sound sensor 120. Using these electrical signals, the environmental classifier 156 is configured to evaluate/analyze the received sound signals (sounds) and determine the sound class/category/environment of the sounds. That is, the environmental classifier 156 is configured to use the received sounds to “classify” the ambient sound environment and/or the sounds into one or more sound categories (i.e., determine the input signal type). The sound class or environment may include, but are not limited to, “Speech” (e.g., the sound signals include primarily speech signals), “Noise” (e.g., the sound signals include primarily noise signals), “Speech+Noise” (e.g., both speech and noise are present in the sound signals), “Wind” (e.g., e.g., the sound signals include primarily wind signals), “Music” (e.g., the sound signals include primarily music signals), and “Quiet” (e.g., the sound signals include minimal speech or noise signals). The environmental classifier 156 may also estimate the signal-to-noise ratio (SNR) of the sounds. In one example, the environmental classifier 156 generates sound classification information/data. The sound classification data represents the sound class of the sound signals and, in certain examples, the SNR of the sound signals.

As noted, sound processing unit 122 also comprises controller 158. The controller 158 is configured to use the sound classification data (e.g., indicating the sound class of the sound signals) to determine whether the implantable acoustic hearing device 100 is in a listening environment in which the target voltage applied to the amplifier 152 could be lowered. For example, if the recipient is in a quiet environment, the target voltage to apply to the amplifier 152 may be adjusted to a level lower than a voltage applied when the recipient is in other types of environments (e.g., louder environments). Lowering the voltage when the recipient is in a quiet environment reduces battery consumption and decreases static noise experienced by the recipient. In such environments, when the environmental classifier identifies that the recipient is in a quiet environment, the controller 158 is configured to dynamically adjust operation of the implantable acoustic hearing device 100 to adjust the power outputted by the at least one battery 132 based on the target voltage required for amplifier 152 to amplify the sound signals.

In some embodiments, an amount of power provided by the at least one battery 132 is adjusted based on the received sound signals regardless of the sound classification data or the environment associated with the recipient. In this embodiment, the target voltage applied to the amplifier 152 is adjusted based on attributes of the received sound signals (e.g., amplitude) and the power supplied by the at least one battery 132 is adjusted based on the target voltage regardless of the sound environment. This could increase maximum acoustic output by using amplifier voltages that would otherwise be prohibited by high static power consumption leading to an unacceptable battery autonomy.

Sound processing unit 122 further includes the interface module 162 that allows the recipient or other user to interact with implantable acoustic hearing device 100. For example, interface module 162 may allow the recipient to adjust the volume, alter the speech processing strategies, power on/off the device, etc. For ease of illustration, interface module 162 has been shown connected only to controller 158.

The processed electrical signals are provided to the RF transceiver 174, which transcutaneously transfers the electrical signals (e.g., in an encoded manner) to the implant body 123 via external coil 108 and implantable coil 180. That is, the electrical signals are received at the RF interface circuitry 140 via implantable coil 180 and provided to the amplifier 152.

In the embodiment illustrated in FIG. 1B, power from the at least one battery 132 is additionally provided to implant body 123 via RF transceiver 174, external coil 108, implantable coil 180, and RF interface circuitry 140. As discussed above, controller 158 may determine the amount of power to provide to implant body 123 may be based on parameters associated with the processed sound signals, such as the amplitude of the processed sound signals. In one example, the amplitude of the processed sound signals is directly proportional to the target voltage to apply to amplifier 152 to drive the actuator 124. In other words, the target voltage to apply to amplifier 152 is approximately the same as the amplitude of the processed sound signals. In some embodiments, controller 158 may determine the target voltage to be higher than the amplitude of the processed sound signals (e.g., 10% higher, 25% higher, etc.). Controller 158 may determine an amount of power to provide to the voltage regulator 160 by the at least one battery 132 to provide the target voltage to the amplifier 152.

In the embodiment illustrated in FIG. 1C, an indication of the power to be provided by the least one battery 132 is additionally provided to implant body 123 via RF transceiver 174, external coil 108, implantable coil 180, and RF interface circuitry 140. In a similar manner as described with respect to FIG. 1B, controller 158 may determine the amount of power to provide based on the target voltage to apply to the amplifier 152. The target voltage may be determined based on the parameters associated with the processed sound signals (e.g., the amplitude). In some embodiments, an indication of the target voltage may additionally be provided from sound processing unit 122 to implant body 123. In some embodiments, RF interface circuitry 140 may output the indication of the power (and optionally the target voltage) to controller 155 and controller 155 may adjust the power provided by the at least one battery 132 to the voltage regulator 160 based on the indication.

The voltage regulator 160 receives the power (e.g., from RF interface circuitry 140 in FIG. 1B or from the at least one battery 132 in FIG. 1C) and supplies a voltage to amplifier 152 to amplify the electrical signals. An amount of the voltage that is supplied is based on an amount of received power. The amplifier 152 amplifies (i.e., increases the time-varying voltage or current) the processed electrical signals to generate amplified output signals. The amplified output signals are provided to actuator 124 via a lead 144 and used to drive (activate) the actuator 124 which, in turn, generates corresponding vibrations. That is, using the amplified output signals, the actuator 124 generates a mechanical output force that is delivered to the skull of the recipient via coupling element 146. As discussed above, the coupling element 146 relays the vibration generated by the actuator 124 to the stapes 114 which, in turn, causes oval window 110 to vibrate. Such vibration of the oval window 110 sets up pressure waves within the cochlea 116 which, in turn, activate hair cells (not shown) that line the inside of the cochlea 116. In some situations (e.g., when the stapes is immobilized or nonexistent), instead of the vibration being relayed to the stapes 114, the actuator 124 may be connected to the skull and the main transmission path to the cochlea 116 is via bone. Activation of these hair cells causes appropriate nerve impulses to be transferred through the spiral ganglion cells and the auditory nerve 118 to the brain (not shown), where they are perceived as sound.

In the embodiment illustrated in FIG. 1A, some components (e.g., sound sensor 120, sound processing module 172, controller 158, etc.) have all been shown as integrated into a single housing. However, it should be appreciated that in certain embodiments of the present invention, one or more of the illustrated components may be housed in separate or different housings. Similarly, it should also be appreciated that in such embodiments, direct connections between the various modules and devices are not necessary and that the components may communicate, for example, via wireless connections.

FIGS. 1A-1C generally illustrate an implantable acoustic hearing device 100 in which the actuator 124 is mechanically coupled to an element of the recipient's middle ear (e.g., the stapes 114). As such, FIGS. 1A-1C generally illustrate an example embodiment in which the actuator 124 is indirectly coupled to the inner ear 103. It is to be appreciated that, in alternative embodiments, an actuator may be directly coupled to the inner ear 103.

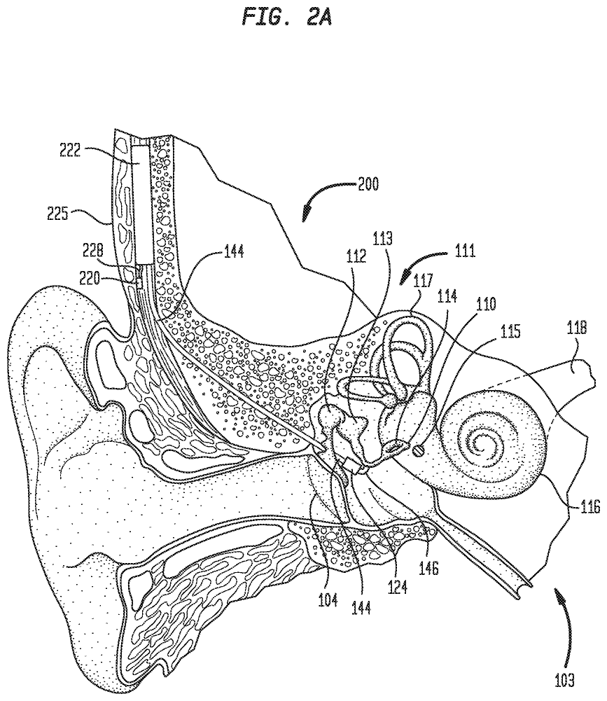

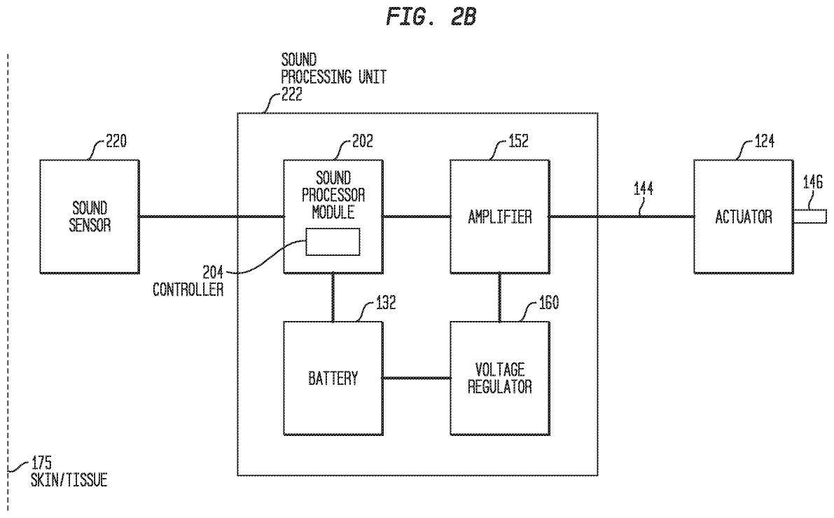

FIG. 2A is a schematic diagram illustrating an implantable acoustic hearing device 200 in accordance with certain embodiments presented herein. FIG. 2B is a block diagram of the implantable acoustic hearing device 200. For ease of description, FIGS. 2A and 2B will be described together.

Similar to the arrangement of FIGS. 1A-1C, the implantable acoustic hearing device 200 is, in general, configured to convert sound signals entering the recipient's outer ear into mechanical vibrations that are directly or indirectly transferred to the cochlea 116, thereby causing generation of nerve impulses that result in the perception of the received sound. The implantable acoustic hearing device 200 comprises a sound sensor 220, a sound processor module 202, at least one battery 132, a voltage regulator 160, an amplifier 152, and an actuator 124 all implanted in the head 225 and under the skin/tissue 175 of the recipient. Since all components of the implantable acoustic hearing device 200 are implanted within the head 225 of the recipient, the implantable acoustic hearing device 200 can be referred to as a “totally-implantable” acoustic hearing device. Although not shown in FIGS. 2A and 2B, the sound sensor 220 and the actuator 124 can each include hermetically-sealed housings.

FIG. 2A illustrates an example in which the sound sensor 220 is a subcutaneous microphone. However, it is to be appreciated that this specific configuration is illustrative and that a variety of different sound sensors and locations may be used in accordance with certain embodiments presented herein.

As shown in FIG. 2B, the sound processing unit 222 comprises a sound processor module 202, at least one battery 132, a voltage regulator 160, and an amplifier 152 each disposed in a hermetically-sealed housing. The at least one battery 132 is configured to supply power to the other components of the implantable acoustic hearing device 200. For ease of illustration, connections between the at least one battery 13 and some of the various powered components of the implantable acoustic hearing device 200 have been omitted from FIG. 2B. It is also to be appreciated that the specific arrangement for sound processing unit 222 shown in FIG. 2B is illustrative and that the sound processing unit 222 could include other elements, such as a radio frequency (RF) coil, RF interface circuitry, a wireless interface, etc. that, for ease of illustration, have been omitted from FIG. 2B.

In the examples of FIGS. 2A and 2B, the sound processor module 202 performs functions similar to functions performed by one or more elements described with respect to FIGS. 1B and 1C that, for ease of illustration, have been omitted (e.g., sound processing module 172, environmental classifier 156, interface module 162, etc.). The sound processor module 202 includes controller 204. Controller 204 performs various functions similar to the functions performed by controllers 155 and 158.

Sound processor module 202 receives electrical signals from the sound sensor 220. In response, the sound processor module 202 processes (e.g., adjusts, amplifies, etc.) the received electrical signals according to the hearing needs of the recipient. That is, the sound processor module 202 converts the electrical signals into processed signals. The processed signals generated by the sound processor module 202 are then provided to the amplifier 152.

In one embodiment, sound processor module 202 determines a target voltage to apply to amplifier 152 based on the processed signals. For example, controller 204 of sound processor module 202 may determine the target voltage based on the amplitude and/or other parameters of the processed signals. Controller 204 may additionally determine an amount of power to be supplied to voltage regulator 160 by the at least one battery 132 for voltage regulator 160 to supply the target voltage to amplifier 152. An indication of the power to be supplied by the at least one battery 132 is transmitted to the at least one battery 132 and the at least one battery 132 provides the indicated amount of power to voltage regulator 160 to output the target voltage to amplifier 152. Amplifier 152 amplifies the processed signals based on the voltage received from voltage regulator 160. The amplified signals are provided to actuator 124 via a lead 144 and are used to activate/actuate or drive the actuator 124 to generate mechanical stimulation (e.g., vibrations for delivery to an auditory element of the hearing anatomy of the recipient) via the coupling element 146.

Collectively, FIGS. 1A-1C, 2A, and 2B illustrate various arrangements for implantable acoustic hearing devices. As noted, these arrangements are merely examples and that the embodiments presented herein may be implemented in totally-implantable acoustic hearing devices, mostly-implantable acoustic hearing devices, semi-implantable acoustic hearing devices, and/or other types hearing devices. In accordance with the various embodiments presented herein, an implantable actuator is either directly or indirectly coupled to the inner ear of recipient to deliver mechanical stimulation (vibration) thereto.

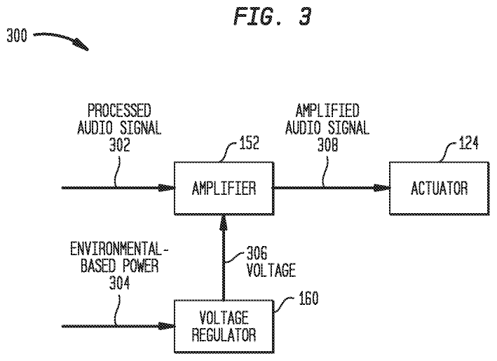

FIG. 3 is a schematic diagram illustrating a portion of an implantable acoustic hearing device 300 in accordance with certain embodiments presented herein. The portion of the implantable acoustic hearing device 300 includes voltage regulator 160, amplifier 152, and actuator 124.

As discussed above, a dynamic power link can be used to supply power to an implantable acoustic hearing device, and a voltage regulator can be provided in the implantable acoustic hearing device to further regulate the voltage supplied to the amplifier. However, in certain sound/ambient environments, the voltage can operate in a lower range than a normal/nominal operating voltage, which saves power and reduces noise. For example, in a quiet environment, the voltage supplied to the amplifier may be relatively lower, which requires less battery power, and reduces the static noise experienced by a recipient of the acoustic hearing device.

As discussed above, when a sound signal is received at an acoustic hearing device (for example, at sound sensor 120 or sound sensor 220), the sound signal is processed to produce a processed audio signal. The target voltage required at the amplifier to drive the actuator may be determined based on parameters of the processed audio signal. In one embodiment, the amplitude of the processed audio signal may be used to determine the voltage to supply to the amplifier to drive the actuator. A one-to-one relationship exists between the voltage and the amplitude. Therefore, in some embodiments, the target voltage to apply to the amplifier 152 may be calculated to be the same as the amplitude of the processed audio signal. In other embodiments, the target voltage may be calculated to be higher than the amplitude of the processed audio signal (e.g., 10% higher, 25% higher, etc.). When the target voltage is determined, the power needed to supply the target voltage to the amplifier may be determined.

In the embodiment illustrated in FIG. 3, amplifier 152 receives processed audio signal 302. In one embodiment, sound processing module 172 (illustrated in FIGS. 1B and 1C) may receive an incoming sound signal from sound sensor 120 (illustrated in FIGS. 1B and 1C) and process the incoming sound signal to produce the processed audio signal 302. Amplifier 152 may receive the processed audio signal 302 from sound processing module 172 over an RF link (e.g., via RF transceiver 174, external coil 108, implantable coil 180, and RF interface circuitry 140). In another embodiment, sound processor module 202 (illustrated in FIG. 2B) may receive an incoming sound signal from sound sensor 220 (illustrated in FIG. 2B) and may process the incoming sound signal to produce the processed audio signal 302. Amplifier 152 may receive the processed audio signal 302 from sound processor module 202.

A target voltage to supply to amplifier 152 may be determined based on the processed audio signal 302. For example, controller 158 (illustrated in FIGS. 1B and 1C) or controller 204 (illustrated in FIG. 2B) may determine the target voltage to supply to amplifier 152 based on properties of the processed audio signal 302. In one embodiment, controller 158/controller 204 may determine the target voltage based on the amplitude of the processed audio signal 302. For example, the target voltage may be equal to the amplitude of the processed audio signal 302. In another example, the target voltage may be greater than the amplitude of the processed audio signal 302 (e.g., 10% greater, 25% greater, etc.).

In one embodiment, controller 158/controller 204 may determine the target voltage when the recipient of the implantable acoustic hearing device is in a particular environment, such as a quiet environment (i.e., environmental classifier 156 has identified the environment as quiet). In this embodiment, when the recipient is in other environments, the voltage supplied to amplifier 152 is 3.3V. In other embodiments, the target voltage to supply to amplifier 152 may be determined regardless of the recipient's environment.

When the target voltage has been determined, controller 158/controller 204 may determine the power to be supplied by the at least one battery 132 to voltage regulator 160 to power the amplifier 152 to the target voltage. As illustrated in FIG. 3, voltage regulator 160 receives environmental-based power 304, which is determined based on the calculated target voltage.

In one embodiment, as illustrated in FIG. 1B, voltage regulator 160 may receive environmental-based power 304 from battery 132 over an RF link when the battery 132 is in an external sound processing unit 122. In this embodiment, controller 158 may determine the amount of power to supply to voltage regulator 160 and the power is transmitted across the RF link to voltage regulator 160. An amount of power supplied may be adjusted based on increasing or decreasing frame length.

In another embodiment, as illustrated in FIG. 1C, voltage regulator 160 may receive environmental-based power 304 from at least one battery 132 located in in implant body 123. In this embodiment, controller 158 may determine the amount of power to supply to voltage regulator 160 and may send an indication of the determined amount of power over the RF link to controller 155. Controller 155 may adjust the amount of power that the at least one battery 132 supplies to voltage regulator 160.

In yet another embodiment, as illustrated in FIG. 2B, voltage regulator 160 may receive environmental-based power 304 from battery 132 in sound processing unit 222. In this embodiment, sound processor module 202 may determine the amount of power to supply to voltage regulator 160 and controller 204 may adjust the amount of power that battery 132 supplies to voltage regulator 160 to supply to target voltage to amplifier 152.

As further illustrated in FIG. 3, voltage regulator 160 receives environmental-based power 304 and provides a voltage 306 to amplifier 152 based on an amount of environmental-based power 304 received. Voltage 306 may be the same as (or nearly the same as) the target voltage calculated based on the processed audio signal 302. Amplifier 152 amplifies the processed audio signal 302 based on the received voltage 306 to provide an amplified audio signal 308 to actuator 124. The actuator 124 generates a mechanical output force that is delivered to the skull of the recipient.

By lowering the voltage supplied to the amplifier 152, battery power may be conserved by supplying enough power to the voltage regulator 160 to power the amplifier 152 to the lower voltage. In addition, since noise is roughly proportional to voltage squared, static noise will also decrease by lowering the voltage supplied to the amplifier 152 in quiet environments when the static noise would be the most audible. The system may be combined with an overriding control loop to make sure implant voltage stays within a low-high limit.

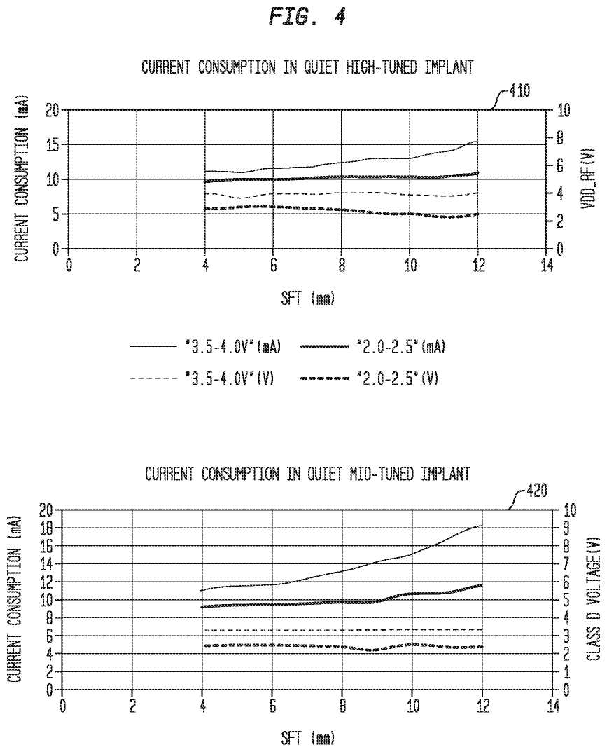

FIG. 4 shows graphs 410 and 420 that illustrate the power savings that may be achieved by lowering the voltage supplied to amplifier 152.

To produce the results illustrated in graphs 410 and 420, dynamic power level parameters were adjusted to 2.2V to 2.5V and the battery current and the amplifier voltages were measured for different skin flap thicknesses (SFT) in millimeters (mm) and in quiet conditions. Two implantable acoustic hearing devices, each with a different tuning, were tested and the voltage and current measurements were recorded. Graph 410 illustrates the results associated with a high-tuned implant and graph 420 illustrates the results associated with the mid-tuned implant.

As illustrated in graphs 410 and 420, the current consumption is decreased significantly for lower voltages supplied to the amplifier. Power savings are the highest for large skin flap thicknesses. The current consumption is decreased by up to 50% for the largest skin flap thicknesses when lowering the voltage.

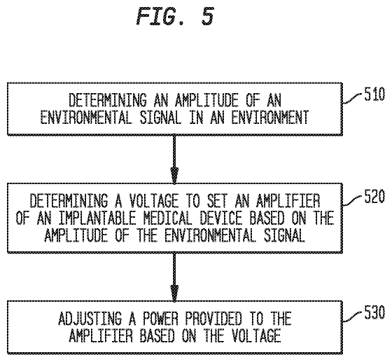

FIG. 5 is a flow diagram illustrating a method 500 of adjusting power supplied to amplifier of an implantable medical device, such as an implantable acoustic hearing device, based on a received environmental/spatial signal (e.g., sound signal).

At 510, method 500 includes determining an amplitude of an environmental signal in an environment. For example, a sound sensor of an implantable medical device (e.g., an implantable acoustic hearing device) may receive an environmental/spatial signal (e.g., sound signal) and the environmental signal may be processed. An amplitude of the processed signal may be determined. In one example, the amplitude may be determined when the environment is identified as a quiet environment.

At 520, method 500 includes determining a voltage to set an amplifier of the implantable medical device based on the amplitude of the environmental signal. For example, a voltage to provide to an amplifier of the implantable acoustic hearing device to amplify the environmental signal to drive an actuator may be determined at a processing module of the implantable acoustic hearing device based on the amplitude of the environmental signal.

At 530, method 500 includes adjusting a power provided to the amplifier based on the voltage. For example, an amount of power to provide to a voltage regulator to supply the determined voltage to an amplifier may be adjusted. For example, the power may be adjusted by adjusting by a frame length.

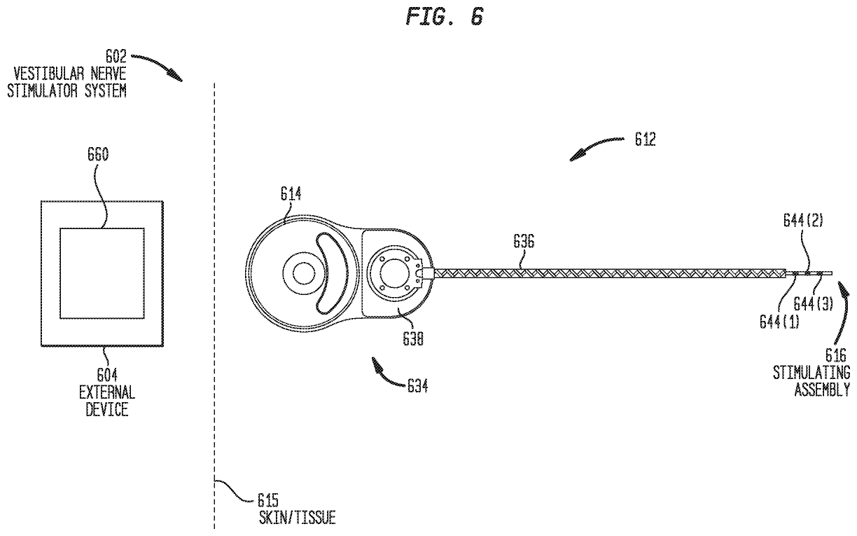

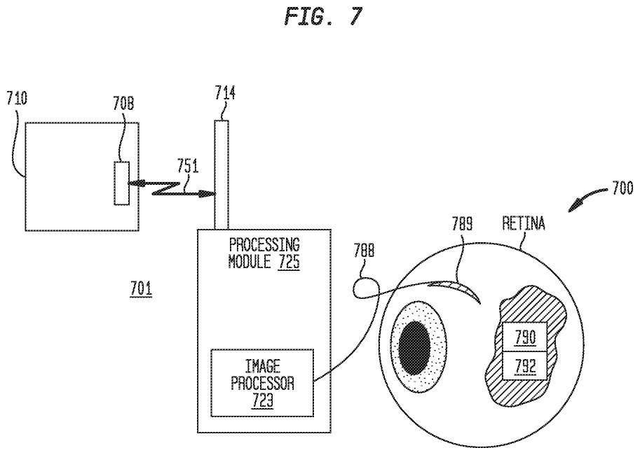

As previously described, the technology disclosed herein can be applied in any of a variety of circumstances and with a variety of different devices. Example devices that can benefit from technology disclosed herein are described in more detail in FIGS. 6 and 7. The techniques of the present disclosure can be applied to other devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 6 illustrates an example vestibular stimulator system 602, with which embodiments presented herein can be implemented. As shown, the vestibular stimulator system 602 comprises an implantable component (vestibular stimulator) 612 and an external device/component 604 (e.g., external processing device, battery charger, remote control, etc.). The external device 604 comprises a transceiver unit 660. As such, the external device 604 is configured to transfer data (and potentially power) to the vestibular stimulator 612, The vestibular stimulator 612 comprises an implant body (main module) 634, a lead region 636, and a stimulating assembly 616, all configured to be implanted under the skin/tissue (tissue) 615 of the recipient. The implant body 634 generally comprises a hermetically-sealed housing 638 in which RF interface circuitry, one or more rechargeable batteries, one or more processors, and a stimulator unit are disposed. The implant body 634 also includes an internal/implantable coil 614 that is generally external to the housing 638, but which is connected to the transceiver via a hermetic feedthrough (not shown).

The stimulating assembly 616 comprises a plurality of electrodes 644(1)-(3) disposed in a carrier member (e.g., a flexible silicone body). In this specific example, the stimulating assembly 616 comprises three (3) stimulation electrodes, referred to as stimulation electrodes 644(1), 644(2), and 644(3). The stimulation electrodes 644(1), 644(2), and 644(3) function as an electrical interface for delivery of electrical stimulation signals to the recipient's vestibular system.

The stimulating assembly 616 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 612, the external device 604, and/or another external device, can be configured to implement the techniques presented herein. That is, the vestibular stimulator 612, possibly in combination with the external device 604 and/or another external device, can include an evoked biological response analysis system, as described elsewhere herein.

FIG. 7 illustrates a retinal prosthesis system 701 that comprises an external device 710 (which can correspond to the implantable acoustic hearing device 100) configured to communicate with an implantable retinal prosthesis 700 via signals 751. The retinal prosthesis 700 comprises an implanted processing module 725 and a retinal prosthesis sensor-stimulator 790 is positioned proximate the retina of a recipient. The external device 710 and the processing module 725 can communicate via coils 708, 714.

In an example, sensory inputs (e.g., photons entering the eye) are absorbed by a microelectronic array of the sensor-stimulator 790 that is hybridized to a glass piece 792 including, for example, an embedded array of microwires. The glass can have a curved surface that conforms to the inner radius of the retina. The sensor-stimulator 790 can include a microelectronic imaging device that can be made of thin silicon containing integrated circuitry that convert the incident photons to an electronic charge.

The processing module 725 includes an image processor 723 that is in signal communication with the sensor-stimulator 790 via, for example, a lead 788 which extends through surgical incision 789 formed in the eye wall. In other examples, processing module 725 is in wireless communication with the sensor-stimulator 790. The image processor 723 processes the input into the sensor-stimulator 790, and provides control signals back to the sensor-stimulator 790 so the device can provide an output to the optic nerve. That said, in an alternate example, the processing is executed by a component proximate to, or integrated with, the sensor-stimulator 790. The electric charge resulting from the conversion of the incident photons is converted to a proportional amount of electronic current which is input to a nearby retinal cell layer. The cells fire and a signal is sent to the optic nerve, thus inducing a sight perception.

The processing module 725 can be implanted in the recipient and function by communicating with the external device 710, such as a behind-the-ear unit, a pair of eyeglasses, etc. The external device 710 can include an external light/image capture device (e.g., located in/on a behind-the-ear device or a pair of glasses, etc.), while, as noted above, in some examples, the sensor-stimulator 790 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

What is claimed is:

1. A method comprising:
determining an amplitude of an environmental signal in an environment;
determining a voltage to set an amplifier of an implantable medical device based on the amplitude of the environmental signal; and
adjusting a power provided to the amplifier based on the voltage.

2. The method of claim 1, wherein adjusting the power comprises:
adjusting the power transmitted from a battery across a wireless link to the amplifier.

3. The method of claim 1, wherein adjusting the power comprises:
adjusting the power provided to the amplifier by an implanted battery.

4. The method of claim 3, wherein adjusting the power comprises:
sending an indication of the power to a controller over a wireless link, wherein the controller is configured to adjust the power provided by the implanted battery.

5. The method of claim 1, wherein adjusting the power comprises adjusting the power transmitted to a voltage regulator that regulates the voltage supplied to the amplifier.

6. The method of claim 1, wherein determining the amplitude of the environmental signal comprises:
determining the amplitude of a sound signal.

7. The method of claim 6, wherein determining the amplitude includes:
determining that the environment is a quiet sound environment; and
determining the amplitude based on the determining that the environment is a quiet sound environment.

8. The method of claim 6, wherein the sound signal is a processed sound signal.

9. The method of claim 8, wherein the voltage to set the amplifier is substantially equal to the amplitude of the processed sound signal.

10. The method of claim 8, wherein the voltage to set the amplifier is greater than the amplitude of the processed sound signal.

11. The method of claim 1, wherein the implantable medical device is an implantable acoustic hearing device comprising an actuator, and wherein the method further comprises:
driving the actuator with amplified output signals generated by the amplifier from the environmental signal.

12. (canceled)

13. One or more non-transitory computer readable storage media comprising instructions that, when executed by a processor, cause the processor to:
determine at least one attribute of a signal captured from an ambient environment;
determine a voltage to set an amplifier of an implantable medical-device based on the at least one attribute of the signal; and
adjust a power provided to the amplifier based on the voltage.

14. The one or more non-transitory computer readable storage media of claim 13, wherein, when adjusting the power, the instructions cause the processor to at least one of:
adjust the power transmitted across a wireless link to the amplifier,
adjust the power provided to the amplifier by an implanted battery, or
adjust the power transmitted to a voltage regulator that regulates the voltage supplied to the amplifier.

15. (canceled)

16. (canceled)

17. (canceled)

18. The one or more non-transitory computer readable storage media of claim 13, wherein the at least one attribute is an amplitude of the signal.

19. (canceled)

20. (canceled)

21. (canceled)

22. (canceled)

23. An implantable medical device comprising:
a battery;
an amplifier; and
at least one processor configured to set a power provided by the battery to the amplifier based on an amplitude of at least one signal captured from an ambient environment of the implantable medical device.

24. The implantable medical device of claim 23, wherein the at least one processor is configured to:
determine an amplitude of the at least one signal in the ambient environment;
determine a voltage to set the amplifier based on the amplitude of the at least one signal; and
adjust a power provided by the battery to the amplifier based on the voltage.

25. The implantable medical device of claim 23, wherein the battery and the at least one processor are in an external portion of the implantable medical device and the amplifier is in an implantable portion of the implantable medical device, and wherein when setting the power, the at least one processor is configured to adjust the power transmitted across a wireless link to the amplifier.

26. The implantable medical device of claim 23, further comprising a controller, wherein the at least one processor is in an external portion of the implantable medical device and the controller, the battery, the amplifier are in an implantable portion of the implantable medical device and wherein, when setting the power, the at least one processor is configured to cause the controller to adjust the power provided to the amplifier by the battery.

27. The implantable medical device of claim 23, further comprising a voltage regulator configured to regulate a voltage supplied to the amplifier, wherein, when setting the power, the at least one processor is further configured to adjust the power provided to the voltage regulator.

28. (canceled)

29. (canceled)

30. (canceled)

31. (canceled)

32. The implantable medical device of claim 23, further comprising:
an actuator,
wherein the amplifier is configured to generate amplified output signals used to drive the actuator to generate vibrations corresponding to the signal.

33. (canceled)
